(19) United States
(12) Patent Application Publication
(10) Pub. No.: US 2012/0129808 A1
(43) Pub. Date: May 24, 2012

(54) BLOOD COAGULATION REACTION INHIBITOR

(75) Inventor: Tomohide Tsuda, Kanagawa (JP)

(73) Assignee: SHINO-TEST CORPORATION, Tokyo (JP)

(21) Appl. No.: 12/737,114

(22) PCT Filed: Jun. 12, 2008
(86) PCT No.: PCT/JP2008/061165

§ 371 Date: Dec. 9, 2010

(51) Int. Cl. A61K 31/685; A61P 7/02

(52) U.S. Cl. 514/77

(57) ABSTRACT

The present invention is a blood coagulation reaction inhibitor comprising phosphatidylserine, phosphatidylcholine, and phosphatidylethanolamine. The present invention is also a thrombin generation inhibitor comprising phosphatidylserine, phosphatidylcholine, and phosphatidylethanolamine. The blood coagulation reaction inhibitor and the thrombin generation inhibitor of the present invention are useful as an antithrombotic agent, a clinical laboratory test reagent for the blood coagulation-fibrinolysis system, and the like.

### TECHNICAL FIELD

The present invention relates to a blood coagulation reaction inhibitor containing a phospholipid, the inhibitor being useful as an antithrombotic agent for treating a disease and in a clinical laboratory test of the blood coagulation-fibrinolysis system and the like.

### BACKGROUND ART

A phospholipid is a major lipid constituting various membrane systems of cells making up an organism, such as a plasma membrane, a nuclear membrane, an endoplasmic reticulum membrane, a mitochondrial membrane, a chloroplastic membrane, and a bacterial cytoplasmic membrane. It is also contained in serum lipids and egg yolks.

Phospholipids are categorized into glycerophospholipids and sphingophospholipids, depending on constituents thereof. Known examples of glycerophospholipids include phosphatidylserine, phosphatidylcholine (lecithin), lysophosphatidylcholine, phosphatidylethanolamine, phosphatidylinositol, phosphatidylglycerol, and diphosphatidylglycerol (cardiolipin). Known examples of sphingophospholipids include sphingomyelin.

When bleeding is caused by injury of a blood vessel, a hemostatic plug sealing the injured vessel is formed to stop the bleeding; this reaction comprises primary thrombosis in which platelets play the leading role and secondary thrombosis occurring as a result of coagulation factor activation. These two reaction systems do not proceed completely independently of each other, but acceleratingly proceed only in local injured sites while influencing each other. It has been known for a long time that some steps of the blood coagulation reaction are promoted in the presence of a phospholipid.

Hemker et al. have reported that in the reactions in which thrombin is generated from prothrombin by activated factor X and the activated factor X is generated from factor X by activated factor IX, a mixture of phosphatidylserine and phosphatidylcholine significantly reduces Km values for the substrates (prothrombin and factor X) for the respective reactions. That is, they have reported that the mixture of phosphatidylserine and phosphatidylcholine has the effect of promoting blood coagulation reaction. They have elucidated that a biomembrane containing a phospholipid has the role of binding to each of the activated coagulation factors (activated factor VIII and activated factor V), a substrate, and a cofactor to increase their local concentration. [See Non-Patent Document 1]

It has been known that protein C is activated by a complex of thrombomodulin and thrombin (a thrombomodulin-thrombin complex) and that phosphatidylethanolamine activates the thrombomodulin-thrombin complex. [Patent Document 1]

However, the inhibition of thrombus formation by the activation of the thrombomodulin-thrombin complex involves activating the reaction of a coagulation control system [the activated factor VIII-hydrolyzing reaction by activated protein C, protein S and calcium ion; and the activated factor V-hydrolyzing reaction by activated protein C, protein S and calcium ion] to hydrolyze the activated factor VIII and the activated factor V as promoters of coagulation reaction, thereby indirectly inhibiting the generation of thrombin, that is, the formation of thrombus. Thus, the effect of activating the coagulation control system cannot be expected in patients having abnormality in the system.

For example, in patients with APC resistance (activated factor V Leiden disease), the amino acid at the point of the activated factor V cleaved by the activated protein C is mutated and thus the activated factor V cannot be completely hydrolyzed by the activated protein C. Hence, even when the generation of the activated protein C is activated, the activated factor V cannot be completely hydrolyzed and thus the coagulation reaction cannot be inhibited; that is, the formation of thrombus cannot probably be inhibited. [Japanese Journal of Clinical Medicine 62 (Extra issue 12): 678-680 (2004)]

Protein S, which is an activator for the activity of the activated protein C, is deficient or significantly reduced in patients having protein S deficiency. The activity of the activated protein C is not sufficiently exhibited in such a state in which protein S as an activator for the activated protein C activity is deficient or significantly reduced; thus, even though the generation of the activated protein C is activated, the activated protein C activity cannot reach a level effective for hydrolyzing the activated factor VIII and the activated factor V, the activated factor VIII and the activated factor V are not sufficiently hydrolyzed, and thus the coagulation reaction cannot be inhibited; that is, the formation of thrombus cannot probably be inhibited. [Japanese Journal of Clinical Medicine 62 (Extra issue 12): 685 (2004)]

In addition, in patients having protein C deficiency, protein C is deficient or significantly reduced. The activity of the activated protein C is not sufficiently exhibited in such a state in which protein S is deficient or significantly reduced; thus, even though the generation of the activated protein C is activated, the activated protein C activity cannot reach a level effective for hydrolyzing the activated factor VIII and the activated factor V, the activated factor VIII and the activated factor V are not sufficiently hydrolyzed, and thus the coagulation reaction cannot be inhibited; that is, the formation of thrombus cannot probably be inhibited. [Journal of Clinical Medicine 62 (Extra issue 12): 684-685 (2004)]
[Patent Document 1]: JP Patent Publication (Kokai) No. 5-58899
[Non-Patent Document 1]: Yutaka Matsumoto and Yasuo Ikeda, Mebio 11 (3): 26-31, Medical View K.K. (1994)

### DISCLOSURE OF THE INVENTION

Thus, an object of the present invention is to provide a blood coagulation reaction inhibitor and a thrombin generation inhibitor useful as an antithrombotic agent and the like.

During the study of the relation between blood coagulation reaction and phospholipid, the present inventors have found that the coexisting of phosphatidylserine, phosphatidylcholine, and phosphatidylethanolamine has the effect of inhibiting the blood coagulation reaction, thereby accomplishing the present invention.

Thus, the present invention provides the following inventions.

(1) A blood coagulation reaction inhibitor comprising phosphatidylserine, phosphatidylcholine, and phosphatidylethanolamine.

(2) The blood coagulation reaction inhibitor of item (1), used as an antithrombotic agent.

(3) The blood coagulation reaction inhibitor of item (1), used as a clinical laboratory test reagent for a blood coagulation-fibrinolysis system.

(4) A thrombin generation inhibitor comprising phosphatidylserine, phosphatidylcholine, and phosphatidylethanolamine.

### EFFECTS OF THE INVENTION

The blood coagulation reaction inhibitor and thrombin generation inhibitor of the present invention have excellent blood coagulation reaction-inhibiting and thrombin generation-inhibiting actions and are useful as an antithrombotic agent, a clinical laboratory test reagent for the blood coagulation-fibrinolysis system, and the like.

The blood coagulation reaction inhibitor and thrombin generation inhibitor of the present invention inhibit thrombus formation by directly inhibiting the blood coagulation system, and are thought to exhibit their effect even in patients having APC resistance, protein S deficiency, or protein C deficiency.

### BRIEF DESCRIPTION OF THE DRAWINGS

FIG. 1 is a graph showing the relation between the amount of phosphatidylethanolamine and the amount of thrombin generated in a blood coagulation reaction for a phospholipid composition comprising phosphatidylserine, phosphatidylcholine, and phosphatidylethanolamine.

FIG. 2 is a graph showing the relation between the amount of phosphatidylethanolamine and the plasma clotting time in a blood coagulation reaction for a phospholipid composition comprising phosphatidylserine, phosphatidylcholine, and phosphatidylethanolamine.

FIG. 3 is a graph showing the average value of the length of discoloration on rat tails in an experiment of administration of a solution of each phospholipid composition to thrombosis model rats.

FIG. 4 is a pair of photographs of the tail of a thrombosis model rat to which a phosphate buffered saline solution is administered.

FIG. 5 is a pair of photographs of the tail of a thrombosis model rat to which a phospholipid composition solution containing phosphatidylserine, phosphatidylcholine, and phosphatidylethanolamine is administered.

FIG. 6 is a pair of photographs of the tail of a thrombosis model rat to which a phospholipid composition solution containing phosphatidylserine and phosphatidylcholine is administered.

FIG. 7 is a pair of photographs of the tail of a thrombosis model rat to which a phospholipid composition solution containing phosphatidylserine and lysophosphatidylcholine is administered.

### BEST MODE FOR CARRYING OUT THE INVENTION

The phosphatidylserine used in the present invention is a generic term applied to glycerophospholipids each containing L-serine phosphate as a polar group. Examples of a hydrophobic group bound to the 1-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

Examples of a hydrophobic group bound to the 2-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

The phosphatidylserine used in the present invention can be used without particular limitation; examples thereof include one isolated from an animal, a plant, a bacterium, or the like before purification, one chemically synthesized, or one prepared by biotechnology. The phosphatidylserine used in the present invention can also be used without particular limitation provided that it is one purified to such an extent that it can be used as a medicine.

The phosphatidylcholine used in the present invention is a generic term applied to glycerophospholipids each containing choline phosphate as a polar group. Examples of a hydrophobic group bound to the 1-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

Examples of a hydrophobic group bound to the 2-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

The phosphatidylcholine used in the present invention is not particularly limited; examples thereof include one isolated from an animal, a plant, a bacterium, or the like before purification, one chemically synthesized, or one prepared by biotechnology. The phosphatidylcholine used in the present invention can also be used without particular limitation provided that it is one purified to such an extent that it can be used as a medicine.

The phosphatidylethanolamine used in the present invention is a generic term applied to glycerophospholipids each containing ethanolamine phosphate as a polar group. Examples of a hydrophobic group bound to the 1-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

Examples of a hydrophobic group bound to the 2-position of glycerol can include saturated or unsaturated fatty acid residues each having 4 to 25 carbons. More specific examples thereof can include acyl groups; still more specific examples thereof can include, as corresponding fatty acids, capric acid, lauric acid, myristic acid, palmitic acid, stearic acid, arachidic acid, behenic acid, palmitoleic acid, oleic acid, elaidic acid, vaccenic acid, erucic acid, linoleic acid, linolenic acid, or arachidonic acid.

The phosphatidylethanolamine used in the present invention can be used without particular limitation; examples thereof include one isolated from an animal, a plant, a bacterium, or the like before purification, one chemically synthesized, or one prepared by biotechnology. The phosphatidylethanolamine used in the present invention can also be used without particular limitation provided that it is one purified to such an extent that it can be used as a medicine.

The blood coagulation reaction inhibitor and thrombin generation inhibitor of the present invention are a phospholipid composition comprising phosphatidylserine, phosphatidylcholine, and phosphatidylethanolamine.

The ratio among phosphatidylserine, phosphatidylcholine, and phosphatidylethanolamine in the phospholipid composition is not particularly limited; however, the mass ratio of phosphatidylserine:phosphatidylcholine:phosphatidylethanolamine is preferably 1:0.1 to 10:0.5 to 10, more preferably 1:0.25 to 7.5:1 to 7.5, particularly preferably 1:0.5 to 5:1.5 to 5. The mass ratio of phosphatidylethanolamine to the total of phosphatidylserine and phosphatidylcholine is typically 0.25 or more, preferably 0.5 to 5.0, more preferably 0.7 to 3.0.

The blood coagulation reaction inhibitor and thrombin generation inhibitor of the present invention can be used as an antithrombotic agent or an ingredient of a clinical laboratory test reagent for the blood coagulation-fibrinolysis system, or the like.

The blood coagulation reaction inhibitor and thrombin generation inhibitor of the present invention can be used in various dosage forms. The dosage form is preferably, for example, a liposomal preparation or a fat emulsion preparation.

The liposomal preparation can be prepared according to a conventional method; for example, it can be prepared by dissolving the above phospholipids in an organic solvent such as chloroform, evaporating off the solvent to dry up the solution, and then, adding purified water or a suitable buffer solution, if necessary, together with a conventional additive such as a stabilizer, followed by sonication.

The fat emulsion preparation can also be prepared according to a conventional method. For example, it can be prepared by adding a fat and oil and an emulsifier to water as well as adding the above phospholipids and, if necessary, adding a conventional additive such as a stabilizer, followed by emulsification.

When the blood coagulation reaction inhibitor or thrombin generation inhibitor of the present invention is used as a therapeutic agent (a medicine) such as an antithrombotic agent, their usage does not matter provided that it is effective. The usage is preferably percutaneous absorption, intravenous administration, or the like. The dosage can be properly adjusted depending on the weight, age, degree of disease, and the like of a patient; however, they may each be typically administered at 0.0001 to 0.1 g/kg body weight/day, preferably 0.001 to 0.01 g/kg body weight/day as the amount of a phospholipid composition once or several times daily in divided portions.

The blood coagulation reaction inhibitor or thrombin generation inhibitor of the present invention can be contained in a reagent for measuring activated protein C or protein C which is a factor involved in the blood coagulation reaction system to promote the measurement reaction, enabling the measurement to be rapidly performed with high sensitivity.

### EXAMPLES

The present invention will now be specifically described below with reference to Examples. However, the present invention is not limited by these Examples.

### Example 1

### Blood Coagulation Reaction-Inhibiting Action of Phospholipid Composition Comprising Phosphatidylserine, Phosphatidylcholine, and Phosphatidylethanolamine

An “activated factor X-prothrombin reaction system” in which blood coagulation factors in the living body were reconstituted was constructed to ascertain the blood coagulation reaction-inhibiting action of a phospholipid composition comprising phosphatidylserine, phosphatidylcholine, and phosphatidylethanolamine.

### 1. Reagents

(1) First Reagent

A 50 mM tris-hydrochloric acid buffer solution (pH 7.5 (20° C.)) was prepared which contains 0.5% (v/v) of each of the following phospholipid composition solutions, 30 μM of activated factor V, 700 nM of prothrombin, 750 μM of S-2238 [a substrate (cromogenic substrate) for thrombin; Daiichi Pure Chemicals Co., Ltd.], 150 mM of sodium chloride, 5 mM of calcium chloride, and 0.1% of bovine serum albumin, and called a first reagent.

The phospholipid composition solutions are listed below.

(a) An aqueous solution containing 0.4 mg/mL of phosphatidylserine and 0.4 mg/mL of phosphatidylcholine.

(b) An aqueous solution containing 0.4 mg/mL of phosphatidylserine, 0.4 mg/mL of phosphatidylcholine, and 0.2 mg/mL of phosphatidylethanolamine.

(c) An aqueous solution containing 0.4 mg/mL of phosphatidylserine, 0.4 mg/mL of phosphatidylcholine, and 0.4 mg/mL of phosphatidylethanolamine.

(d) An aqueous solution containing 0.4 mg/mL of phosphatidylserine, 0.4 mg/mL of phosphatidylcholine, and 0.6 mg/mL of phosphatidylethanolamine.

(e) An aqueous solution containing 0.4 mg/mL of phosphatidylserine, 0.4 mg/mL of phosphatidylcholine, and 0.8 mg/mL of phosphatidylethanolamine.

(f) An aqueous solution containing 0.4 mg/mL of phosphatidylserine, 0.4 mg/mL of phosphatidylcholine, and 1.2 mg/mL of phosphatidylethanolamine.

The phosphatidylserine, phosphatidylcholine, and phosphatidylethanolamine used the following products.

(Phosphatidylserine)

L-α-Phosphatidylserine, Bovine Brain, Funakoshi Reagent General Catalog 2004 part II, p. 919, manufacturer: DOOSAN Serdary Research Laboratories, Commodity Code: A-37

(Phosphatidylcholine)

L-α-Phosphatidylcholine, Porcine Liver, Funakoshi Reagent General Catalog 2004 part II, p. 907, manufacturer: DOOSAN Serdary Research Laboratories, Commodity Code: A-29, purity: 98 to 99%

(Phosphatidylethanolamine)

L-α-Phosphatidylethanolamine, Porcine Liver, Funakoshi Reagent General Catalog 2004 part II, p. 914, manufacturer: DOOSAN Serdary Research Laboratories, Commodity Code: A-33, purity: 98 to 99%

(2) Second Reagent

A 50 mM tris-hydrochloric acid buffer solution (pH 7.5 (20° C.)) was prepared which contains 0.5% (v/v) of each of the following phospholipid composition solutions, 100 μM of activated factor X, 150 mM of sodium chloride, 5 mM of calcium chloride, and 0.1% of bovine serum albumin, and called a second reagent.

The phospholipid composition solutions are listed below.

(a) An aqueous solution containing 0.4 mg/mL of phosphatidylserine and 0.4 mg/mL of phosphatidylcholine.

(b) An aqueous solution containing 0.4 mg/mL of phosphatidylserine, 0.4 mg/mL of phosphatidylcholine, and 0.2 mg/mL of phosphatidylethanolamine.

(c) An aqueous solution containing 0.4 mg/mL of phosphatidylserine, 0.4 mg/mL of phosphatidylcholine, and 0.4 mg/mL of phosphatidylethanolamine.

(d) An aqueous solution containing 0.4 mg/mL of phosphatidylserine, 0.4 mg/mL of phosphatidylcholine, and 0.6 mg/mL of phosphatidylethanolamine.

(e) An aqueous solution containing 0.4 mg/mL of phosphatidylserine, 0.4 mg/mL of phosphatidylcholine, and 0.8 mg/mL of phosphatidylethanolamine.

(f) An aqueous solution containing 0.4 mg/mL of phosphatidylserine, 0.4 mg/mL of phosphatidylcholine, and 1.2 mg/mL of phosphatidylethanolamine.

### 2. Operating Procedure

The first reagent (360 μL) was added into a cuvette of a spectrophotometer and warmed at 37° C. for 5 minutes, to which the second reagent (180 μL) warmed in advance at 37° C. was then added, followed by warming at 37° C. for reaction.

Here, the activated factor X converts prothrombin to thrombin in the presence of the activated factor V. Then, the generated thrombin hydrolyzes the chromogenic substrate S-2238 to release p-nitroaniline. The p-nitroaniline has absorption maximum around the wavelength of 405 nm.

The measurement and recording of the absorbance of the solution in the cuvette at the wavelength of 405 nm were carried out every one minute from 5 minutes before the addition of the second reagent until 30 minutes thereafter.

Known concentrations of thrombin and the chromogenic substrate S-2238 were reacted with each other, and a calibration curve was made from the relation between the thrombin concentration and the generation rate of p-nitroaniline.

Then, the amount of change in absorbance per minute at the wavelength of 405 nm when the phospholipid composition having each composition was used was fitted to the calibration curve to determine the generation rate (nM/min.) of thrombin, that is, the rate of the blood coagulation reaction.

### 3. Measurement Result

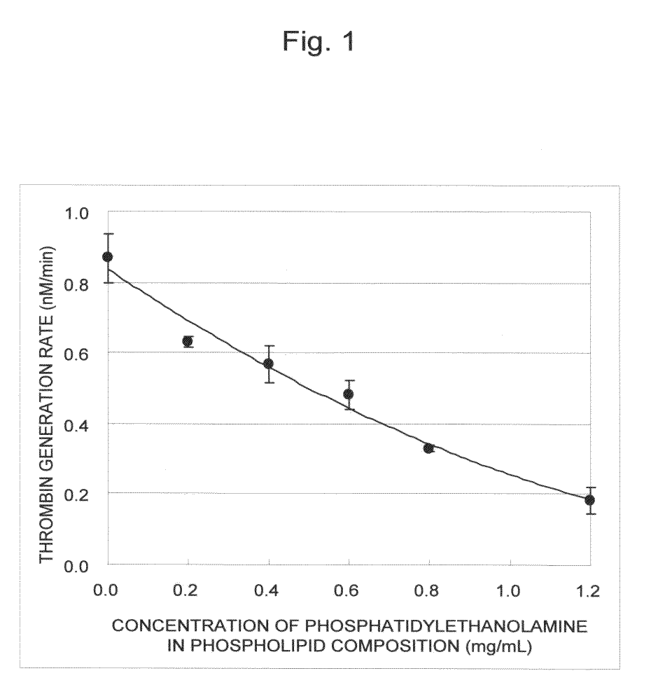

The measurement results are shown in FIG. 1.

In the figure, the horizontal axis represents the concentration (mg/mL) of phosphatidylethanolamine in the phospholipid compositions, and the vertical axis represents the generation rate (nM/min.) of thrombin.

This figure shows that the addition of phosphatidylethanolamine reduces the generation rate of thrombin, that is, reduces the rate of the blood coagulation reaction, compared to that for only phosphatidylserine and phosphatidylcholine.

It also shows that the generation rate of thrombin (the rate of the blood coagulation reaction) decreases with increasing phosphatidylethanolamine concentration.

These results confirmed that the phospholipid composition comprising phosphatidylserine, phosphatidylcholine, and phosphatidylethanolamine had the action of inhibiting the blood coagulation reaction.

### Example 2

### Blood Coagulation Reaction-Inhibiting Action of Phospholipid Composition Comprising Phosphatidylserine, Phosphatidylcholine, and Phosphatidylethanolamine

The plasma clotting time in the blood coagulation reaction was measured to ascertain the blood coagulation reaction-inhibiting action of a phospholipid composition comprising phosphatidylserine, phosphatidylcholine, and phosphatidylethanolamine.

(1) Operating Procedure

Human plasma (50 μL) was warmed at 37° C. for one minute, to which a solution (50 μL) obtained by diluting each of the following phospholipid composition solutions to 2% (v/v) with a 50 mM tris-hydrochloric acid buffer solution (pH 7.5 (20° C.)) was then added, followed by warming at 37° C. for 2 minutes. After the end of warming, 50 μL of 20 mM calcium chloride was added, and the time which it takes for the plasma in the mixed solution to be coagulated was measured.

The phospholipid composition solutions are listed below.

(a) An aqueous solution containing 0.4 mg/mL of phosphatidylserine and 0.4 mg/mL of phosphatidylcholine.

(b) An aqueous solution containing 0.4 mg/mL of phosphatidylserine, 0.4 mg/mL of phosphatidylcholine, and 0.2 mg/mL of phosphatidylethanolamine.

(c) An aqueous solution containing 0.4 mg/mL of phosphatidylserine, 0.4 mg/mL of phosphatidylcholine, and 0.6 mg/mL of phosphatidylethanolamine.

(d) An aqueous solution containing 0.4 mg/mL of phosphatidylserine, 0.4 mg/mL of phosphatidylcholine, and 1.2 mg/mL of phosphatidylethanolamine.

(e) An aqueous solution containing 0.4 mg/mL of phosphatidylserine, 0.4 mg/mL of phosphatidylcholine, and 2.0 mg/mL of phosphatidylethanolamine.

The phosphatidylserine, phosphatidylcholine, and phosphatidylethanolamine used the following products.

(Phosphatidylserine)

L-α-Phosphatidylserine, Bovine Brain, Funakoshi Reagent General Catalog 2004 part II, p. 919, manufacturer: DOOSAN Serdary Research Laboratories, Commodity Code: A-37

(Phosphatidylcholine)

L-α-Phosphatidylcholine, Porcine Liver, Funakoshi Reagent General Catalog 2004 part II, p. 907, manufacturer: DOOSAN Serdary Research Laboratories, Commodity Code: A-29, purity: 98 to 99%

(Phosphatidylethanolamine)

L-α-Phosphatidylethanolamine, Porcine Liver, Funakoshi Reagent General Catalog 2004 part II, p. 914, manufacturer: DOOSAN Serdary Research Laboratories, Commodity Code: A-33, purity: 98 to 99%

(2) Measurement Result

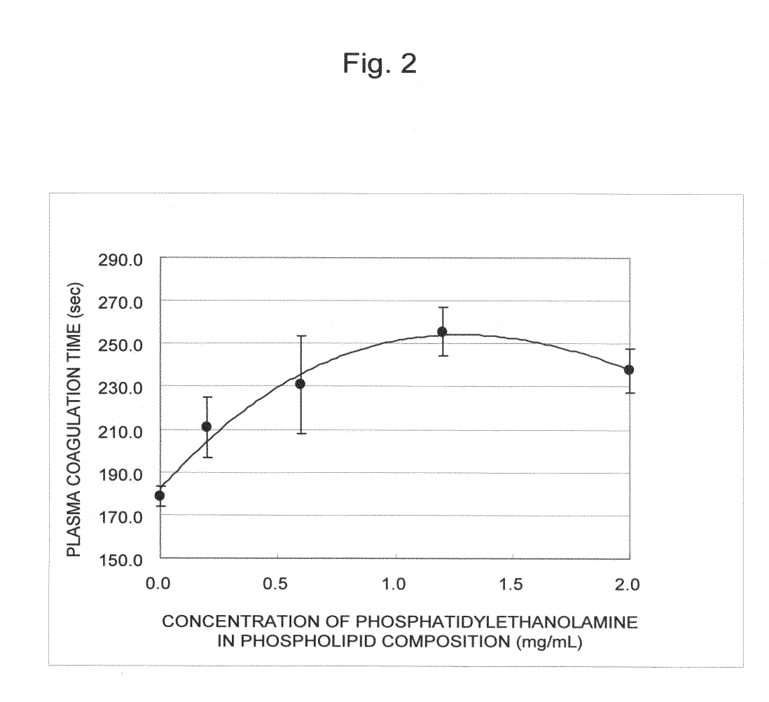

The measurement results are shown in FIG. 2.

In the figure, the horizontal axis represents the concentration (mg/mL) of phosphatidylethanolamine in the phospholipid compositions, and the vertical axis represents the plasma clotting time (seconds).

The plasma clotting time when the phospholipid composition is not added at measurement was 213 seconds.

The measurement results show that the addition of phosphatidylethanolamine increases the plasma clotting time, that is, reduces the rate of the blood coagulation reaction, compared to that for only phosphatidylserine and phosphatidylcholine.

The results also show that the plasma clotting time increases (the rate of the blood coagulation reaction reduces) with increasing phosphatidylethanolamine concentration.

These confirmed that the phospholipid composition comprising phosphatidylserine, phosphatidylcholine, and phosphatidylethanolamine had the action of inhibiting the blood coagulation reaction.

### Example 3

### Blood Coagulation Reaction-Inhibiting Action of Phospholipid Composition Comprising Phosphatidylserine, Phosphatidylcholine, and Phosphatidylethanolamine

Thrombosis model rats were used to ascertain the action of inhibiting the blood coagulation reaction, that is, the effect of preventing thrombosis, of a phospholipid composition comprising phosphatidylserine, phosphatidylcholine, and phosphatidylethanolamine.

### 1. Preparation of Phospholipid Composition Solution

(1) Preparation of PBS

Sterilized phosphate buffered saline [pH 7.4] (“PBS”) was prepared as a control.

(2) Preparation of PS/PC/PE Solution

(a) Phosphatidylserine (1.2 mg), phosphatidylcholine (1.2 mg), and phosphatidylethanolamine (3.6 mg) were added to, mixed with, and dissolved in 3 mL of a 4:1 mixture of chloroform and methanol in a pear-shaped flask.

(b) Nitrogen gas was then injected into the pear-shaped flask to evaporate the mixture of chloroform and methanol.

(c) Thereafter, nitrogen gas was injected into the pear-shaped flask, which was then allowed to stand in a drying box for about 2 hours.

(d) Next, 3 mL of sterilized phosphate buffered saline [pH 7.4] (PBS) was added to the pear-shaped flask, which was then subjected to sonication at reflux under nitrogen gas for 10 minutes.

(e) After the sonication, the pear-shaped flask was filled with nitrogen gas, which was then subjected to chilled storage.

In this manner, a phosphate buffered saline solution comprising 0.4 mg/mL of phosphatidylserine, 0.4 mg/mL of phosphatidylcholine, and 1.2 mg/mL of phosphatidylethanolamine (“PS/PC/PE solution”) was prepared.

(3) Preparation of PS/PC Solution

(a) Phosphatidylserine (5.4 mg) and phosphatidylcholine (0.6 mg) were added to, mixed with, and dissolved in 3 mL of a 4:1 mixture of chloroform and methanol in a pear-shaped flask.

(b) Subsequent operations were carried out according to the description of (b) to (e) in the above “(2) Preparation of PS/PC/PE Solution” to prepare a phosphate buffered saline solution comprising 1.8 mg/mL of phosphatidylserine and 0.2 mg/mL of phosphatidylcholine (“PS/PC solution”).

(4) Preparation of PS/LPC Solution

(a) Phosphatidylserine (5.4 mg) and lysophosphatidylcholine (0.6 mg) were added to, mixed with, and dissolved in 3 mL of a 4:1 mixture of chloroform and methanol in a pear-shaped flask.

(b) Subsequent operations were carried out according to the description of (b) to (e) in the above “(2) Preparation of PS/PC/PE Solution” to prepare a phosphate buffered saline solution comprising 1.8 mg/mL of phosphatidylserine and 0.2 mg/mL of lysophosphatidylcholine (“PS/LPC solution”).

The above phosphatidylserine, phosphatidylcholine, phosphatidylethanolamine, and lysophosphatidylcholine used the following products.

(Phosphatidylserine)

L-α-Phosphatidyl-L-serine, NOF Corporation, trade name: COATSOME MS-818LS

(Phosphatidylcholine)

L-α-Phosphatidylcholine, NOF Corporation, trade name: COATSOME MC-8080

(Phosphatidylethanolamine)

L-α-Phosphatidylethanolamine, NOF Corporation, trade name: COATSOME NE-8080

(Lysophosphatidylcholine)

L-α-Lysophosphatidylcholine, Wako Pure Chemical Industries Ltd. (MP Biomedical; Ohio, US), trade name: 1,3-Lysolecithin, Commodity Code: 100058

### 2. Experiment of Administration of Phospholipid Composition Solution to Thrombosis Model Rats

(1) Administration of PBS

(a) PBS prepared in the (1) of 1 was subcutaneously injected in an amount of 50 μL into each of two spots on each of the places 1 cm, 2 cm, 3 cm, 4 cm, and 5 cm away from the base of the tail of each rat. This operation was carried out in a total of 4 rats.

(b) Twelve hours after administration of PBS to the rats in the (a), the rats were anesthetized and their tails were each tied with a kite string (tourniquet). Then, a 1 mg/mL carrageenin solution was intravenously injected into the tail of each rat.

(c) Ten minutes after intravenous injection, the kite string (tourniquet) with which the tail was tied was cut.

(d) The tail of each rat was observed for discoloration (i.e., change in color) and loss 2 hours, 4 hours, 6 hours, 8 hours, 24 hours, 48 hours, and 72 hours after the cutting.

(e) The length of discoloration (change in color) on the tail of each rat 6 hours, 24 hours and 48 hours after the cutting of the kite string (tourniquet) was shown in Table 1.

TABLE 1
Length (cm) of Discoloration (Change
in Color) on Tail of Each Rat

| | 6 Hours | 24 Hours | 48 Hours |
|---|---|---|---|
| PBS | 13.0 | 12.5 | 12.7 |
| | 12.7 | 12.8 | 13.0 |
| | 12.8 | 13.0 | 12.6 |
| | 12.7 | 12.2 | 12.4 |
| | 12.8 ± 0.1 | 12.6 ± 0.2 | 12.7 ± 0.1 |
| PS/PC/PE | 11.7 | 11.9 | 11.7 |
| Solution | 9.0 | 9.2 | 9.4 |
| | 8.2 | 8.4 | 8.4 |
| | 12.7 | 12.4 | 12.0 |
| | 8.5 | 8.3 | 8.3 |
| | 10.0 ± 0.9 | 10.0 ± 0.9 | 10.0 ± 0.8 |
| PS/PC | 11.5 | 11.4 | 11.4 |
| Solution | 11.5 | 11.5 | 11.6 |
| | 13.2 | 13.4 | 12.0 |
| | 13.3 | 12.0 | 11.8 |
| | 13.1 | 12.3 | 12.4 |
| | 12.7 | 12.5 | 12.6 |
| | 12.6 ± 0.3 | 12.2 ± 0.3 | 12.0 ± 0.2 |
| PS/LPC | 13.0 | 12.8 | 12.9 |
| Solution | 13.0 | 12.5 | 12.2 |
| | 13.3 | 13.3 | 13.4 |
| | 13.1 ± 0.1 | 12.9 ± 0.2 | 12.8 ± 0.4 |

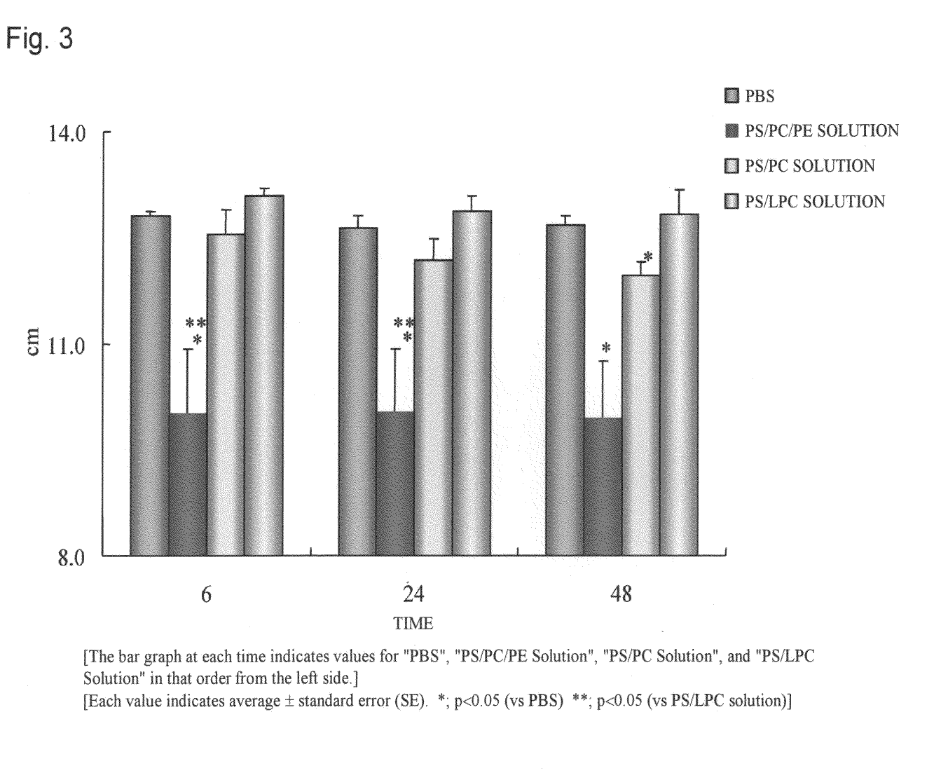

The average values of the length of discoloration (change in color) on the tail of each rat 6 hours, 24 hours and 48 hours after the cutting of the kite string (tourniquet) were shown in FIG. 3. In FIG. 3, the bar graph at each time indicates values for “PBS”, “PS/PC/PE Solution”, “PS/PC Solution”, and “PS/LPC Solution” in that order from the left side.

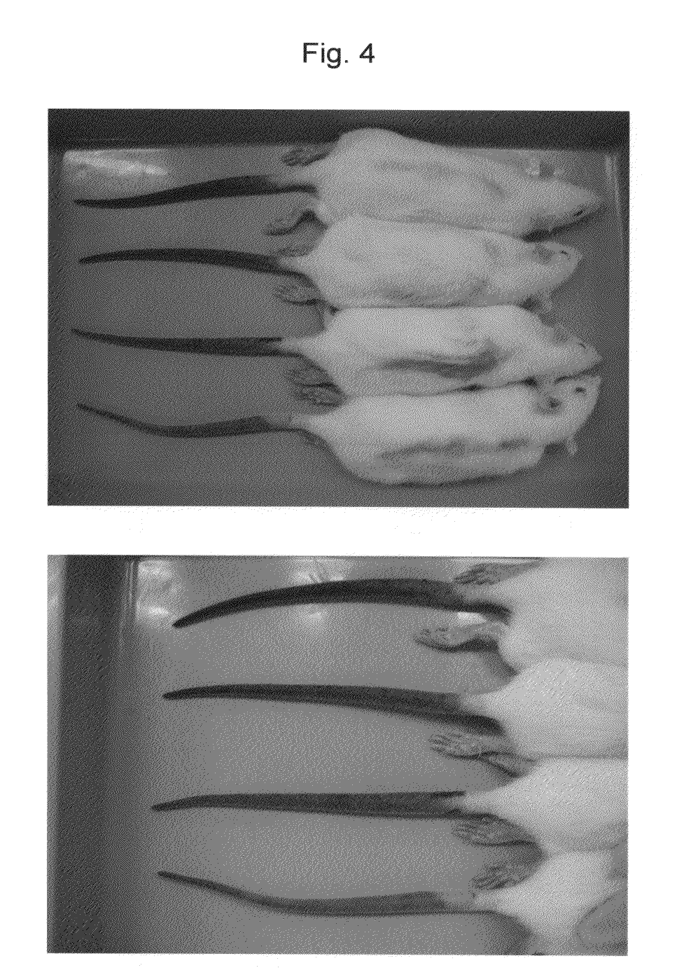

Photographs of the tails of the rats 24 hours after cutting the kite string (tourniquet) were shown in FIG. 4.

(2) Administration of PS/PC/PE Solution

(a) The PS/PC/PE solution prepared in the (2) of 1 was subcutaneously injected in an amount of 50 μL into each of two spots on each of the places 1 cm, 2 cm, 3 cm, 4 cm, and 5 cm away from the base of the tail of each rat. This operation was carried out in a total of 5 rats.

(b) Subsequent operations were carried out according to the description of (b) to (d) in the above “(1) Administration of PBS”.

(c) The length of discoloration (change in color) on the tail of each rat 6 hours, 24 hours and 48 hours after the cutting of the kite string (tourniquet) was shown in Table 1.

The average values of the length of discoloration (change in color) on the tail of each rat 6 hours, 24 hours and 48 hours after the cutting of the kite string (tourniquet) were shown in FIG. 3.

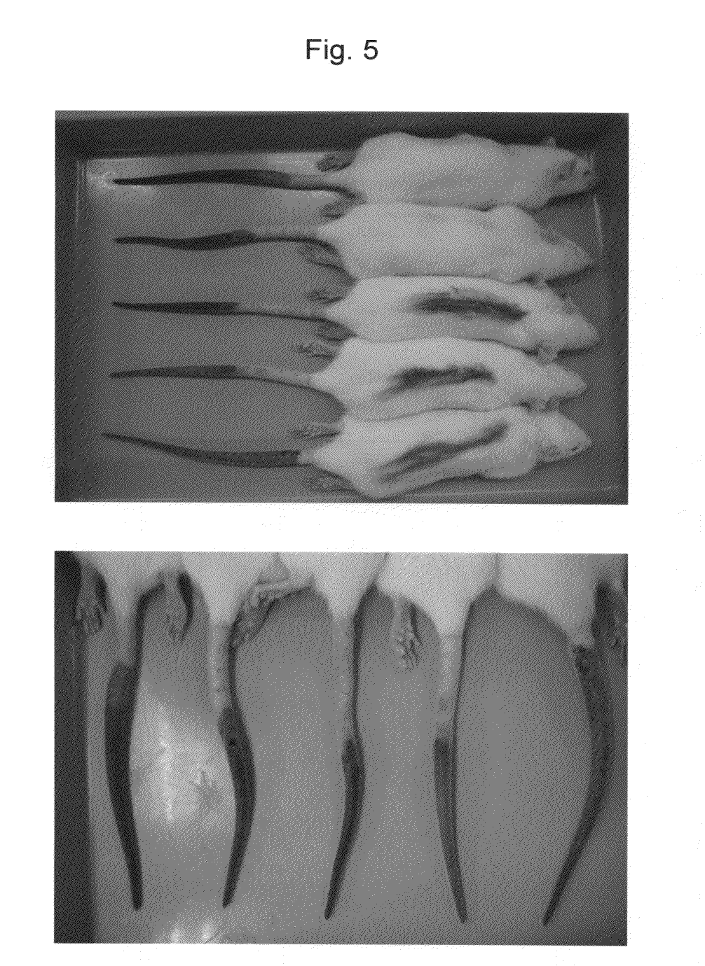

Photographs of the tails of the rats 24 hours after cutting the kite string (tourniquet) were shown in FIG. 5.

(3) Administration of PS/PC Solution

(a) The PS/PC solution prepared in the (3) of 1 was subcutaneously injected in an amount of 50 μL into each of two spots on each of the places 1 cm, 2 cm, 3 cm, 4 cm, and 5 cm away from the base of the tail of each rat. This operation was carried out in a total of 6 rats.

(b) Subsequent operations were carried out according to the description of (b) to (d) in the above “(1) Administration of PBS”.

(c) The length of discoloration (change in color) on the tail of each rat 6 hours, 24 hours and 48 hours after the cutting of the kite string (tourniquet) was shown in Table 1.

The average values of the length of discoloration (change in color) on the tail of each rat 6 hours, 24 hours and 48 hours after the cutting of the kite string (tourniquet) were shown in FIG. 3.

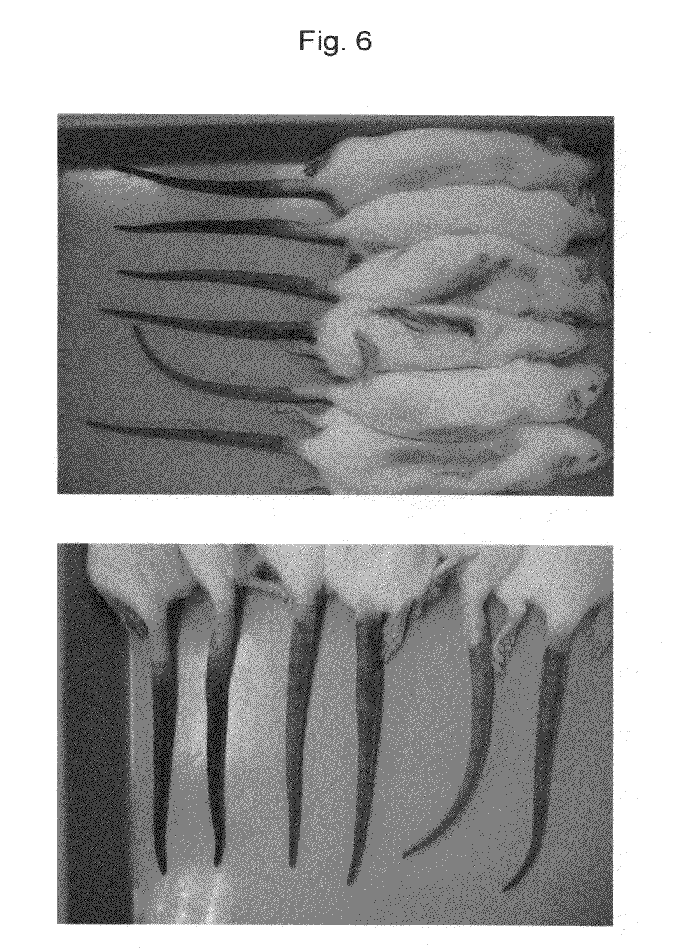

Photographs of the tails of the rats 24 hours after cutting the kite string (tourniquet) were shown in FIG. 6.

(4) Administration of PS/LPC Solution

(a) The PS/LPC solution prepared in the (4) of 1 was subcutaneously injected in an amount of 50 μL into each of two spots on each of the places 1 cm, 2 cm, 3 cm, 4 cm, and 5 cm away from the base of the tail of each rat. This operation was carried out in a total of 3 rats.

(b) Subsequent operations were carried out according to the description of (b) to (d) in the above “(1) Administration of PBS”.

(c) The length of discoloration (change in color) on the tail of each rat 6 hours, 24 hours and 48 hours after the cutting of the kite string (tourniquet) was shown in Table 1.

The average values of the length of discoloration (change in color) on the tail of each rat 6 hours, 24 hours and 48 hours after the cutting of the kite string (tourniquet) were shown in FIG. 3.

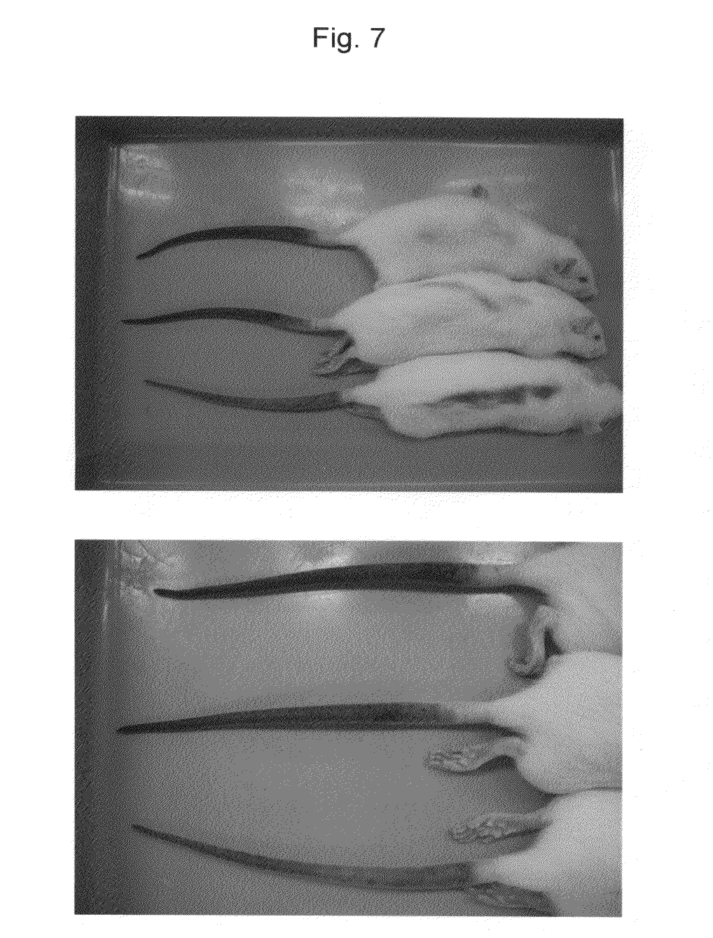

Photographs of the tails of the rats 24 hours after cutting the kite string (tourniquet) were shown in FIG. 7.

### 3. Summary

The results of experiment of administration of each phospholipid composition solution to thrombosis model rats shown in Table 1 and FIGS. 3 to 7 show that the length of discoloration (change in color) due to thrombus on the tails of the rats given the PS/PC/PE solution was evidently decreased compared to the length of the discoloration (change in color) for the rats given PBS as a control.

In contrast, it is shown that the rats given the PS/PC solution and the rats given the PS/LPC solution were not different in the length of the tail discoloration (change in color) from the rats given PBS as a control.

From these findings, it could be also confirmed in the animal experiment that the blood coagulation reaction inhibitor of the present invention comprising phosphatidylserine, phosphatidylcholine, and phosphatidylethanolamine is excellent in the effect of inhibiting the blood coagulation reaction and useful as an antithrombotic agent.

1. A blood coagulation reaction inhibitor comprising phosphatidylserine, phosphatidylcholine, and phosphatidylethanolamine.

2. The blood coagulation reaction inhibitor of claim 1, used as an antithrombotic agent.

3. The blood coagulation reaction inhibitor of claim 1, used as a clinical laboratory test reagent for a blood coagulation-fibrinolysis system.

4. A thrombin generation inhibitor comprising phosphatidylserine, phosphatidylcholine, and phosphatidylethanolamine.

5. A method for inhibiting blood coagulation comprising contacting blood or plasma with a composition comprising phosphatidylserine, phosphatidylcholine, and phosphatidylethanolamine.

6. The method of claim 5 comprising an effective amount of the composition to a subject in need thereof.

7. A method for inhibiting thrombin generation comprising contacting blood or plasma with a composition comprising phosphatidylserine, phosphatidylcholine, and phosphatidylethanolamine.

8. The method of claim 7 comprising an effective amount of the composition to a subject in need thereof.

9. The blood coagulation reaction inhibitor of claim 1, wherein the mass ratio of phosphatidylserine:phosphatidylcholine:phosphatidylethanolamine is 1:0.1 to 10:0.5 to 10.
